(19) United States
(12) Patent Application Publication
(10) Pub. No.: US 2010/0178338 A1
(43) Pub. Date: Jul. 15, 2010

(54) STABILIZED PHARMACEUTICAL COMPOSITIONS COMPRISING ATORVASTATIN

(75) Inventors: Swati Aggarwal, Delhi (IN); Romi Barat Singh, Varanasi (IN)

Correspondence Address: Ranbaxy Inc., Intellectual Property Department, 600 College Road East, PRINCETON, NJ, 08540, US

(73) Assignee: RANBAXY LABORATORIES LIMITED, New Delhi (IN)

(21) Appl. No.: 12/663,593

(22) PCT Filed: Jun. 11, 2008
(86) PCT No.: PCT/IB2008/052317

§ 371 Date: Jan. 20, 2010

(30) Foreign Application Priority Data
Jun. 11, 2007 (IN) 1246/DEL/2007

(51) Int. Cl. A61K 31/40; A61K 9/20; A61K 9/28; A61P 7/00

(52) U.S. Cl. 424/465; 514/423

(57) ABSTRACT

This invention relates to stabilized pharmaceutical compositions comprising atorvastatin or pharmaceutically acceptable salts thereof, and processes for the preparation of the same.

### FIELD OF THE INVENTION

This invention relates to stabilized pharmaceutical compositions comprising atorvastatin or pharmaceutically acceptable salts thereof, and processes for preparation of the same.

### BACKGROUND OF THE INVENTION

Although cholesterol is an indispensable component of all cell membranes as well as a precursor of a variety of steroid hormones and bile acids, excessively high levels of blood cholesterol and lipids increase the risk of the onset of atherosclerosis and coronary heart disease. The blood cholesterol pool is generally dependent upon dietary uptake of cholesterol and the biosynthesis of cholesterol. HMG-CoA reductase enzyme inhibitors, such as atorvastatin, bring about a reduction in the levels of blood cholesterol, especially the low-density lipoproteins, by inhibiting the synthesis of cholesterol. They are therefore excellent candidates for controlling blood cholesterol levels.

Atorvastatin, which is an inhibitor of the enzyme 3-hydroxy-3-methyl glutaryl coenzyme A reductase (HMG-CoA reductase), is commercially available for the treatment of primary hypercholesterolemia, dysbetalipoproteinemia and homozygous familial hypercholesterolemia.

Various attempts have been made to stabilize atorvastatin. WO 2000/35425 discloses attempts to stabilize statin formulations using buffering agents capable of providing a pH in the range from 7 to 11.

U.S. Pat. Nos. 5,686,104 and 6,126,971 disclose oral pharmaceutical formulations of atorvastatin in which the formulation is described as being stabilized by the addition of a pharmaceutically acceptable alkaline earth metal salt. According to these patents, large amounts of alkaline earth metal salt are required to stabilize the formulation. For example, these patents provide examples in which the drug compositions contain approximately 22% of an alkaline earth metal salt used to stabilize the atorvastatin. Nonetheless, these patents claim and/or state that between 5% and 75% of the composition can be the alkaline earth metal salt. The alkaline earth metal salt is described as providing effective control of the microenvironment of the composition.

WO 2003/068191 discloses a pharmaceutical composition of atorvastatin comprising alkali metal salt additives.

WO 2006/070248 discloses a stabilized pharmaceutical composition of atorvastatin in combination with amlodipine.

### SUMMARY OF THE INVENTION

We have now developed an alternate formulation of atorvastatin comprising mannitol and/or pregelatinized starch as diluent. The formulation was found to be stable during the entire shelf life.

Hence, in one aspect, there is provided a stabilized pharmaceutical composition of atorvastatin for oral administration comprising

a) atorvastatin or pharmaceutically acceptable salts thereof; and

b) a diluent comprising mannitol or pregelatinized starch.

In another aspect, there is provided a stabilized pharmaceutical composition of atorvastatin for oral administration comprising:

a) atorvastatin or pharmaceutically acceptable salts thereof; and

b) a diluent comprising a combination of pregelatinized starch and mannitol.

In another aspect, there is provided a stabilized pharmaceutical composition of atorvastatin for oral administration comprising:

a) atorvastatin or pharmaceutically acceptable salts thereof; and

b) a diluent comprising a combination of pregelatinized starch and mannitol

wherein concentration of mannitol is about 5 to about 30% and the concentration of pregelatinized starch is about 5 to about 15% by weight of the composition.

In another aspect, there is provided a stabilized pharmaceutical composition of atorvastatin for oral administration comprising:
a) from about 5 to about 50% of atorvastatin or pharmaceutically acceptable salts thereof;
b) from about 5 to about 30% of mannitol;
c) from about 5 to about 15% of pregelatinized starch;
d) from about 1 to about 30% of an alkanizing agent;
e) from about 0.01 to about 5% of an antioxidant; and
f) from about 1 to about 10% of a surfactant.

In another aspect, there is provided a process for the preparation of a stabilized pharmaceutical composition of atorvastatin for oral administration comprising combining

a) atorvastatin or pharmaceutically acceptable salts thereof; and

b) a diluent comprising a combination of pregelatinized starch and mannitol.

In another aspect, there is provided a process for the preparation of a stabilized pharmaceutical composition of atorvastatin for oral administration comprising the steps of:
a) dissolving an antioxidant in an organic solvent;
b) spraying the solution of step a) onto pregelatinized starch and optionally other pharmaceutically acceptable inert excipients and drying;
c) blending atorvastatin or pharmaceutically acceptable salts thereof with pharmaceutically acceptable inert excipients;
d) blending mannitol with pharmaceutically acceptable inert excipients; and
e) blending the blends of step b), c) and d) with pharmaceutically acceptable inert excipients,
f) lubricating the blend of step e) and compressing into suitable size tablets or filling into capsules.

In another aspect, there is provided a method of treating primary hypercholesterolemia, dysbetalipoproteinemia and homozygous familial hypercholesterolemia comprising orally administering to a patient, stabilized pharmaceutical composition of atorvastatin comprising:

a) atorvastatin or pharmaceutically acceptable salts thereof; and

b) a diluent comprising a combination of pregelatinized starch and mannitol.

### DETAILED DESCRIPTION OF THE INVENTION

As used herein the term “atorvastatin” refers to include atorvastatin and pharmaceutically acceptable salts thereof such as calcium, magnesium, potassium etc. Atorvastatin may exist in any of the solid state forms available such as amorphous or crystalline polymorphic forms. Pharmaceutical composition of atorvastatin may be used for treatment or prevention of primary hypercholesterolemia, dysbetalipoproteinemia and homozygous familial hypercholesterolemia. Atorvastatin can be present in the composition in an amount up to 50% by weight of the composition.

Atorvastatin can be, for example, milled to obtain a mean particle size d₉₀ of, for example, less than or equal to about 200 μm. This size is obtained either directly through the synthesis or by using conventional milling techniques, such as air jet milling, ball milling, cad milling, multi milling and other suitable size reduction techniques. The particle size of the atorvastatin can be reduced to particle size d₉₀ of, for example, less than or equal to about 200 μm, and more particularly to particle size of between approximately 5 μm and 50 μm. The size of the particles may be analyzed using a conventional particle size analyzer (e.g., a Malvern Master Sizer).

The term “stabilized” as used herein means amount of total related substance is not more than 5%, particularly not more than 4.75%, after a sample has been subjected to stability studies at 40° C. and 75% RH for 3 months. The total related substance includes impurities such as oxo atorvastatin, atorvastatin diepoxide, dihydroxy epoxide and diketoepoxide. The total oxidative impurities which includes oxo atorvastatin and atorvastatin diepoxide is not more than 3.25% after a sample has been subjected to 40° C. and 75% RH for 3 months.

Mannitol is a naturally occurring sugar alcohol having a cool taste compared to sucrose or lactose. It is non-hygroscopic, chemically inert and does not undergo the Maillard reaction, and therefore does not discolor in the presence of free amines. Mannitol is available as powder and free flowing granules, and is used widely in pharmaceutical preparations. The granular form is particularly useful in direct compression technique of preparing tablets. Some of the commercial grades are Mannogem®, Pearlitol® and Partech M®. The mannitol can be present in the concentration from about 5-30% by weight of the composition.

Pregelatinized starch is a starch that has been previously gelatinized and dried to powder form. Pregelatinized starch serves multipurposes, as diluent, binder and disintegrant. Pregelatinized starch may be used herein as a diluent in combination with mannitol, although it may contribute additionally as a disintegrant or binder in the composition. Pregelatinized starch is known to be useful for moisture-sensitive drugs, as it binds readily to moisture. Commercially available grades such as Starch 1500 may be used. The pregelatinized starch can be present in an amount ranging from about 5-15% by weight of the composition.

Properties such as the non-hygroscopic nature of mannitol and binding of pregelatinized starch to moisture, make these components suitable for drugs such as atorvastatin.

The term “pharmaceutical composition” can include solid dosage forms such as tablets, capsules, pills and the like.

The term “pharmaceutically acceptable inert excipient” as used herein includes substances known in the art as diluents, binders, disintegrants, stabilizers, surfactants and lubricants/glidants. The excipients are selected based on the desired physical aspects of the final tablets; e.g., obtaining a tablet with desired hardness and friability, being rapidly dispersible or being easily swallowed, for example.

The term “stabilizer”, as used herein, means an agent that stabilizes a statin, for example, alkanizing agents, chelating agents, photoprotectants or antioxidants.

Examples of suitable antioxidants can include, for example, butylated hydroxyanisole (BHA), sodium ascorbate, butylated hydroxytoluene (BHT), sodium sulfite, propyl gallate, tocopherol, citric acid, malic acid, ascorbic acid or mixtures thereof. The antioxidants can be present at concentrations of, for example, from about 0.01% to about 5% by weight. The antioxidants may be dissolved in organic solvent such as ethanol, isopropanol, n-propanol, acetone, ethyl acetate and mixtures thereof and sprayed on to pharmaceutically acceptable inert excipients. Examples of chelating agents can include, for example, disodium EDTA, edetic acid, citric acid, and combinations thereof. The chelating agents can be present at a concentration of up to approximately 10% by weight of the composition, for example, from about 0.01 to about 5% by weight. The term “photoprotectant” as used herein means an agent for protection from the chemical or physical effects of light on a statin formulation. Examples can include metal oxides such as, for example, titanium oxide, ferric oxide or zinc oxide. The photoprotectant can be present at a concentration of up to approximately 10% by weight of the composition, for example, from about 0.01 to about 5% by weight. The alkanizing agents as used herein can include alkali metal salt additives or alkaline earth metal salt additives. Alkali metal salt additives can be, for example, sodium carbonate, sodium hydroxide, sodium silicate, disodium hydrogen orthophosphate, sodium aluminate and other suitable alkali metal salts. In particular, the alkali metal salt additive can be sodium carbonate or disodium hydrogen orthophosphate. Alkaline earth metal salt additives can include, for example, calcium carbonate, calcium hydroxide, magnesium carbonate, magnesium hydroxide, magnesium silicate, magnesium aluminate, or aluminum magnesium hydroxide. The amount of alkanizing agent may vary from about 1 to about 30% by weight of the composition.

Examples of suitable binders can include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth and sodium alginate. In addition to mannitol and pregelatinized starch, the composition may further comprise additional diluent such as cellulose powdered, microcrystalline cellulose, dextrates, dextrins, dextrose, fructose, kaolin, lactitol, lactose, sorbitol, starch, sucrose, and sugar compressible. Examples of suitable disintegrants can include croscarmellose sodium, starch, crospovidone and sodium starch glycolate. Examples of suitable surfactants include polysorbate 80, polyoxyethylene sorbitan, polyoxyethylene-polyoxypropylene copolymer, and sodium lauryl sulphate. The concentration of surfactant may vary from about 1-10% by weight of the composition. Examples of lubricants and glidants include magnesium stearate, sodium stearyl fumarate, colloidal anhydrous silica, stearic acid, magnesium stearate, calcium stearate, talc, hydrogenated castor oil, sucrose esters of fatty acid, microcrystalline wax, yellow beeswax, white beeswax, and the like. Other suitable binders, diluents, disintegrants, surfactants, lubricants, or glidants would be known to those of skill in the art.

The pharmaceutical composition may be further film coated with functional or non functional layer. The coating may be selected from amongst one or more of those suitable coating materials known in the art. For example, the coating material can be Opadry or Opadry AMB (aqueous moisture barrier). Coating may be performed by applying one or more film forming polymers, with or without other pharmaceutically inert excipients, as a solution/suspension using any conventional coating technique known in the art, such as spray coating in a conventional coating pan or fluidized bed processor; or dip coating. Coating may further comprise coloring agents, for example, coating can include any FDA approved colors for oral use.

The pharmaceutical composition may be packaged in unit dosage pack such as blister or into multiunit dosage pack such as bottle. The bottle may be an oxygen permeable container such as HDPE bottle or oxygen impermeable container such as polyethylene and ethylene vinyl alcohol or glass bottle. The packaging may further comprise oxygen absorbers or desiccants.

The following examples illustrate the invention but do not limit the scope of the invention.

### EXAMPLES

### Example 1

| Ingredients | Qty (in mg) |
|---|---|
| Atorvastatin Calcium equivalent to | 82.88 |
| Atorvastatin 80 mg | |
| Mannitol | 80.00 |
| Pregelatinized starch | 50.00 |
| Microcrystalline cellulose | 441.14 |
| Sodium carbonate anhydrous | 25.00 |
| Butylated Hydroxy Anisole (BHA) | 4.58 |
| Butylated Hydroxy Toluene (BHT) | 0.40 |
| Hydroxypropyl cellulose - low viscosity | 16.00 |
| Colloidal silicon dioxide | 16.00 |
| Croscarmellose sodium | 48.00 |
| Sodium lauryl sulphate | 24.00 |
| Magnesium stearate | 12.00 |
| Isopropyl alcohol | q.s. |
| Coating | |
| Opadry AMB | 32.00 |

### Procedure

1. Butylated hydroxyanisole and butylated hydroxytoluene were dissolved in isopropyl alcohol.
2. About ¾^{th} quantity of microcrystalline cellulose, pregelatinized starch and colloidal silicon dioxide were passed through a screen and transferred to a rapid mixer granulator.
3. The solution of step 1 was added to the bulk of step 2 and mixed.
4. The wet mass was dried at 40° C.-45° C. in a fluidized bed drier and passed through a sieve.
5. Sodium carbonate was milled and passed through a sieve.
6. The remaining ¼^{th} quantity of microcrystalline cellulose was passed through a multimill.
7. Atorvastatin calcium, sodium lauryl sulphate and hydroxypropylcellulose were passed through the screen of a quadro comil.
8. Mannitol and croscarmellose sodium were passed together through a screen.
9. The blends of step 5-7 were added to the blend of step 8 and mixed together.
10. The blend of step 4 was added to the blend of step 9 and mixed together.
11. The blend of step 10 was lubricated with magnesium stearate and compressed into tablets.
12. The compressed tablets of step 11 were coated with a dispersion of Opdray AMB in water.

Material prepared according to Example 1 was subjected to stability studies at 40° C. and 75% RH for 3 months and the total related substance was found to be less than 4.75% and oxidative impurities were found to be less than 3.25%.

### Example 2

| Ingredients | Qty (in mg) |
|---|---|
| Atorvastatin calcium equivalent to | 84.39 |
| atorvastatin 80 mg | |
| Mannitol | 80.00 |
| Pregelatinized starch | 50.00 |
| Microcrystalline cellulose | 455.63 |
| Sodium carbonate anhydrous | 25.00 |
| Butylated Hydroxy Anisole (BHA) | 4.58 |
| Butylated Hydroxy Toluene (BHT) | 0.40 |
| Hydroxypropyl cellulose - low viscosity | 16.00 |
| Croscarmellose sodium | 48.00 |
| Sodium lauryl sulphate | 24.00 |
| Magnesium stearate | 12.00 |
| Isopropyl alcohol | q.s. |
| Coating | |
| Opadry AMB | 32.00 |

### Procedure:

1. Butylated hydroxyanisole and butylated hydroxytoluene were dissolved in isopropyl alcohol.
2. About ¾^{th} quantity of microcrystalline cellulose and pregelatinized starch were passed through a screen and transferred to a rapid mixer granulator.
3. The solution of step 1 was added to the bulk of step 2 and mixed.
4. The wet mass was dried at 40° C.-45° C. in a fluidized bed drier and passed through a sieve.
5. Sodium carbonate was milled and passed through a sieve.
6. The remaining ¼^{th} quantity of microcrystalline cellulose was passed through a multimill.
7. Atorvastatin calcium, sodium lauryl sulphate and hydroxypropylcellulose were passed through the screen of a quadro comil.
8. Mannitol and croscarmellose sodium were passed together through a screen.
9. The blends of step 5-7 were added to the blend of step 8 and mixed together.
10. The blend of step 4 was added to the blend of step 9 and mixed together.
11. The blend of step 10 was lubricated with magnesium stearate and compressed into tablets.
12. The compressed tablets of step 11 were coated with a dispersion of Opdray AMB in water.

We claim:

1. A stabilized pharmaceutical composition of atorvastatin for oral administration comprising
a) atorvastatin or pharmaceutically acceptable salts thereof; and
b) a diluent comprising a combination of pregelatinized starch and mannitol.

2. The pharmaceutical composition according to claim 1 wherein mannitol is present in concentration range of about 5 to about 30% by weight of the composition.

3. The pharmaceutical composition according to claim 1 wherein pregelatinized starch is present in an amount about 5 to about 15% by weight of the composition.

4. The pharmaceutical composition according to claim 1, wherein the atorvastatin is in an amorphous form.

5. The pharmaceutical composition according to claim 1, wherein the pharmaceutical composition further comprises one or more pharmaceutically acceptable inert excipients, selected from diluents, binders, disintegrants, stabilizers, surfactants or lubricants/glidants.

6. The pharmaceutical composition according to claim 5, wherein stabilizers are selected from alkanizing agents, chelating agents, photoprotectants, antioxidants or mixtures thereof.

7. The pharmaceutical composition according to any of the preceding claim, wherein the composition comprises:
a) from about 5 to about 50% of amorphous atorvastatin or pharmaceutically acceptable salts thereof;
b) from about 5 to about 30% of mannitol;
c) from about 5 to about 15% of pregelatinized starch;
d) from about 1 to about 30% of an alkanizing agent;
e) from about 0.01 to about 5% of an antioxidant; and
f) from about 1 to about 10% of a surfactant.

8. The pharmaceutical composition according to any of the preceding claims, wherein the composition is prepared by a process comprising:
a) dissolving an antioxidant in an organic solvent;
b) spraying the solution of step a) on to pregelatinized starch and optionally pharmaceutically acceptable inert excipients and drying;
c) blending atorvastatin with pharmaceutically acceptable inert excipients;
d) blending mannitol with pharmaceutically acceptable inert excipients;
e) blending the blends of step b), c) and d) with pharmaceutically acceptable inert excipients; and
f) lubricating the blend of step e) and compressing or filling the blend into a suitable dosage form

9. The pharmaceutical composition according to claim 8, wherein the organic solvent is selected from of ethanol, isopropanol, n-propanol, acetone, ethyl acetate and mixtures thereof.

10. The pharmaceutical composition according to claim 8, wherein in step b), pregelatinized starch is blended with a binder and a glidant.

11. The pharmaceutical composition according to claim 8, wherein in step c), atorvastatin is further blended with a binder and a surfactant.

12. The pharmaceutical composition according to claim 8, wherein the blend of step e) is compressed into a tablet.

13. The pharmaceutical composition according to claim 12, wherein the tablet is further coated with a nonfunctional coating.

14. The pharmaceutical composition according to claim 1, for the treatment or prevention of primary hypercholesterolemia, dysbetalipoproteinemia or homozygous familial hypercholesterolemia.
